(19) United States
(12) Patent Application Publication
(10) Pub. No.: US 2007/0054291 A1
(43) Pub. Date: Mar. 8, 2007

(54) System for detection of nucleic acids

(76) Inventors: John R. Van Camp, San Ramon, CA (US); Seth R. Stern, Palo Alto, CA (US)

Correspondence Address: KOLISCH HARTWELL, P.C., 200 PACIFIC BUILDING, 520 SW YAMHILL STREET, PORTLAND, OR, 97204, US

(21) Appl. No.: 11/438,959

(22) Filed: May 22, 2006

Related U.S. Application Data
(60) Provisional application No. 60/683,059, filed on May 20, 2005.

(51) Int. Cl. C12Q 1/68; C12M 1/34

(52) U.S. Cl. 435/6; 435/287.2

(57) ABSTRACT

System, including methods, apparatus, and kits, for detection of nucleic acids.

### CROSS-REFERENCE TO RELATED APPLICATION

This application is based upon and claims the benefit under 35 U.S.C. § 119(e) of U.S. Provisional Patent Application Ser. No. 60/683,059, filed May 20, 2005, which is incorporated herein by reference in its entirety for all purposes.

### INTRODUCTION

The genetic material of organisms provides nucleic acid markers that can identify and/or characterize the organisms with high accuracy and sensitivity. For example, nucleic acid markers from microorganisms can be used to identify pathogen contamination of food, air, or water and/or a pathogen infection in an animal. In addition, nucleic acid markers from human genetic material can be used to identify or characterize particular individuals. Nucleic acid detection thus has widespread potential in a growing number of areas, including pathogen detection in the food industry, clinical diagnostics, forensics, and/or biodefense, among others. However, most technologies for detecting nucleic acids rely on relatively expensive and/or complex instrumentation. Accordingly, nucleic acid detection is not available to consumers on-site for routine analysis.

### SUMMARY

The present teachings provide a system, including methods, apparatus, and kits, for detection of nucleic acids.

### BRIEF DESCRIPTION OF THE DRAWINGS

FIG. 1 is a schematic view of an exemplary system for detection of nucleic acids using an optical element with an optical characteristic that changes in response to contact with a nucleic acid analyte, in accordance with aspects of the present teachings.

FIG. 2 is another schematic view of the system of FIG. 1.

FIG. 3 is a somewhat schematic sectional view of an exemplary vessel for contacting an optical element with a sample being tested for a nucleic acid analyte, in accordance with aspects of the present teachings.

FIG. 4 is a somewhat schematic sectional view of another exemplary vessel for contacting an optical element with a sample being tested for a nucleic acid analyte, in accordance with aspects of the present teachings.

FIG. 5 is a somewhat schematic sectional view of an exemplary configuration for contacting an optical element with a sample being tested for a nucleic acid analyte, in accordance with aspects of the present teachings.

FIG. 6 is a somewhat schematic sectional view of another exemplary configuration for contacting an optical element with a sample being tested for a nucleic acid analyte, in accordance with aspects of the present teachings.

FIG. 7 is a schematic view of an exemplary method of detecting nucleic acids with increased sensitivity using an optical element with an optical characteristic that changes in response to contact with a nucleic acid analyte, in accordance with aspects of the present teachings.

FIG. 8 is a plan view of an exemplary device for performing the method of FIG. 7, in accordance with aspects of the present teachings.

FIG. 9 is a plan view of another exemplary device for performing the method of FIG. 7, in accordance with aspects of the present teachings.

FIG. 10 is an exploded view of the device of FIG. 9, taken generally at “10” in FIG. 9.

### DESCRIPTION OF VARIOUS EMBODIMENTS

The present teachings provide a system, including methods, apparatus, and kits, for detection of nucleic acids. The apparatus can include an optical element configured as a hologram (a holographic element), for example, generally as a diffraction grating. The optical element can have an optical characteristic, such as a modulated complex index of refraction or diffraction efficiency, that changes in response to contact with a nucleic acid analyte. In particular, the optical element can include a probe, and particularly an analog probe with reduced charge character (e.g., a peptide nucleic acid), that binds to the nucleic acid analyte. This binding can result in a change in a physical property of the optical element, such as its volume. The optical characteristic can be related to the physical property, such that the change in the physical property induced by binding of the nucleic acid analyte to the probe results in a detectable change in the optical characteristic. Changes in the optical characteristic can be detected as a modification of incident light, such as a change in the direction of propagation, spectrum (e.g., distribution of wavelengths or frequencies), intensity (e.g., total intensity and/or distribution of intensity(ies)), polarization (e.g., direction of electric field), and/or phase (e.g., coherence) of the light after interaction with the optical element. In some examples, the change can be visible by eye, for example, a change in the perceived directional output or color of the optical element, allowing simplification of the apparatus.

The system of the present teachings can provide substantially increased sensitivity for nucleic acid detection. In some examples, use of an analog probe with a reduced negative charge can provide a substantially greater change in the optical characteristic than a conventional nucleic acid probe upon binding the nucleic acid analyte. Furthermore, in some examples, the system can am plify the nucleic acid analyte, so that analyte copies (in addition to original analyte) can contact the probe and/or optical element.

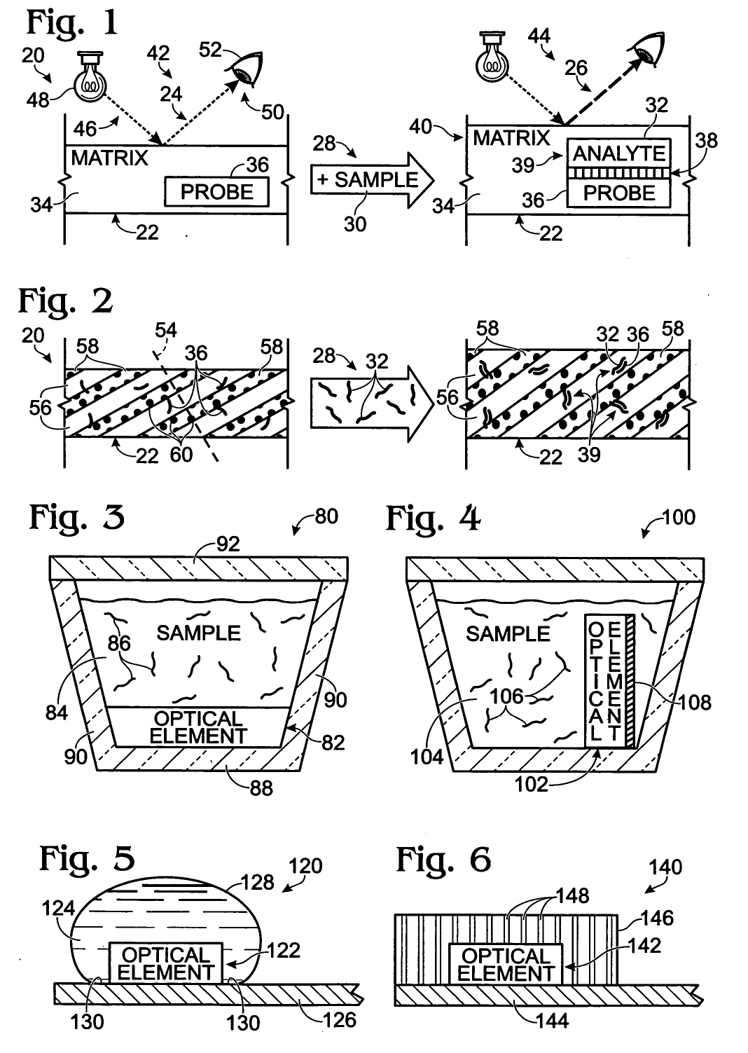

FIG. 1 shows an exemplary system 20 for detection of nucleic acids. The system can include an optical element 22 with an optical characteristic, shown schematically at 24. The optical characteristic can change, shown schematically at 26, in response to contact, indicated at 28, with a sample 30 being tested for a nucleic acid analyte 32 that the optical element is configured to bind selectively. The optical element can be a hologram (a holographic element). Hologram, as used herein, generally refers to a pattern produced on a suitable medium, such as a photosensitive medium, that has been exposed by holography and then photographically developed. For example, the hologram can comprise a photograph of an interference pattern, produced by illuminating the object with a laser or other suitable light source, which contains information about the intensity and phase of light reflected by the object. The resulting hologram, when illuminated at the correct angle with a suitable (e.g., sufficiently coherent) source, will yield a diffracted wave that is at least substantially identical in amplitude and phase distribution with the light reflected from the original object, thereby producing a three-dimensional image of the object.

Optical element 22 can include a matrix 34 and a probe 36. The matrix can contain a fluid, generally a liquid, such that the optical element is structured as a gel. The probe can be disposed in the matrix and retained therein covalently and/or noncovalently. The probe can be configured to bind the nucleic acid analyte selectively, shown at 38, generally by base-pairing to form a double-stranded duplex 39 (and/or duplex region).

The optical characteristic can be affected by a physical property of the optical element. For example, the volume of the optical element can change, shown at 40 (such as by uptake of adjoining fluid to swell the optical element or release of internal fluid to shrink the optical element), to alter an optical characteristic of the optical element.

The optical characteristic can be detected, shown at 42 and 44, before, during, and/or after contact with the sample. Detection can include exposing, shown at 46, the optical element to light from a light source 48 (illumination of the optical element). Detection also can include detecting, shown at 50, interaction of the optical element with the light from the light source using an optical sensor, such as with an eye(s) 52 and/or an artificial sensor element, such as a photodiode, a spectrophotometer, or film, among others. Illumination and sensing can be from the same side of the optical element (e.g., epi-detection or reflection), as shown here, from opposing sides (e.g., trans-detection or transmission), from a side and an end, respectively (or vice versa), and/or the like.

FIG. 2 shows another representation of system 20. Optical element 22 can be composed of an optically heterogeneous material. For example, the optical element can have a local optical character (such the tendency to refract, reflect, absorb, transmit, and/or diffract light), that varies within the optical element. Variation can be patterned, for example, variation can be in a periodic or cyclical fashion along a line 54 through the optical element, such that a numerical measure of the local optical character increases, decreases, increases, decreases, and so on, respectively, along the line. In the present illustration, the optical element has a refractive index that varies along line 54. Regions or layers (or bands) of decreased refractive index, shown at 56, can be interspersed with regions or bands of increased refractive index, shown at 58. The regions of decreased and increased refractive index can be a physical record of fringes of an interference pattern. Furthermore, the regions of increased and decreased reflectivity can be created in the optical element by particles, such as grains 60 of varying size, number (per unit volume (density)), and/or chemical composition, as with development of a silver halide-based photographic emulsion. The development can be followed by fixing and/or bleaching. Accordingly, the grains in the optical element can be, for example, of elemental silver to create an amplitude hologram, or, for example, of silver halide to create a phase hologram.

Contact with sample that includes analyte 32, shown at 28, can change a physical property and an associated optical characteristic of the optical element. For example, in the present illustration, entry of the analyte into the optical element causes the optical element to swell, such that the spacing between refractive minima 56 and between refractive maxima 58 (and between adjacent minima and maxima) increases. Binding between the analyte and probe, to form duplexes 39, retains the analyte in the optical element.

Further aspects of the present teachings are described in the following sections, including (I) optical elements; (II) nucleic acids, including (A) analytes and (B) probes; (III) contacting optical elements with samples; (IV) detecting optical characteristics of optical elements; and (V) examples.

I. Optical Elements

The optical elements of the present teachings can have any suitable structure, including any suitable shape and size, matrix, fluid composition, probe, physical properties, optical characteristics, and/or fabrication methods, among others.

An optical element can have any suitable shape and size. Exemplary shapes for optical elements include a sheet or plate having a length and width that are substantially greater than the thickness of the sheet. The sheet can have any suitable shape when viewed toward a face of the sheet, including circular, polygonal, elliptical, oval, curvilinear, etc. The sheet can have any suitable dimensions. For example, the sheet can be large enough to be visible by eye, without magnification, such that it may be detected and/or viewed by eye and/or suitable optics. Accordingly, the sheet can have a length and/or width of at least about 0.5 mm or about 1 mm, among others. Alternatively, the length and/or width can be less than about 0.5 mm, for detection and/or viewing with suitable optics. The sheet can have any suitable thickness, such as less than about 100, 50, or 20 micrometers. In some examples, the optical element can have a shape distinct from a sheet, such as a shape with a thickness that varies over the surface of the element (e.g., semi-spherical, furrowed, polyhedral, etc.), and/or with a thickness that is not substantially less than the width and/or length.

The matrix of an optical element can have any suitable composition. The matrix can be formed, for example, of a porous framework that contains fluid to create a gel. The framework can include a plurality of subunits that are bonded covalently to one another, as in a polymer, and/or associated noncovalently, as in subunits held together by ionic interactions, hydrogen bonds, van der Waals forces, and/or the like. In some examples, the framework can include ions, particularly multivalent ions, such as calcium or chromium, that bridge two or more subunits. Exemplary matrices that can be suitable include matrices formed of protein (e.g., collagen (including denatured and/or degraded forms thereof, such as gelatin)), carbohydrate (e.g., agarose, alginate, chitosan, starch, etc.), small organic subunits (e.g., to form polyacrylates such as polyacrylamide), polynucleotides, and/or inorganic materials (such as silica), among others. Further aspects of matrices, also termed support media, that can be suitable are described in the following patent, which is incorporated herein by reference: U.S. Pat. No. 5,989,923, to Lowe et al., issued Nov. 23, 1999.

The matrix can hold any suitable fluid. The fluid can be a gas, but is preferably a liquid. Exemplary liquids that can be contained by the matrix include water and/or organic solvents (such as dimethylformamide, dimethylsulfoxide, an alcohol, polyethyleneglycol (PEG), and/or the like). The liquids further may include any suitable soluble and/or suspended materials, such as ions, buffers, cofactors, and so on.

The matrix also can contain or include any other suitable materials. The materials can include optical components, such as particles or dyes, that interact with light, such as by refraction, reflection, polarization, absorption, and/or fluorescence, among others. The optical components can have a nonuniform (or uniform) distribution within the optical element. The optical components can be concentrated in layers disposed within the optical elements. In some examples, the layers can be generally parallel to one another. The layers can be arranged generally parallel to faces of the optical element, obliquely to the faces, and/or substantially perpendicular to the faces. The materials also or alternatively can include one or more probes for selectively binding particular nucleic acid analytes. The probes can be attached covalently to the matrix and/or can associate with the matrix noncovalently, such as by specific binding pair interactions (e.g., receptor-ligand, biotin-avidin, antibody-antigen, etc.). Further aspects of probes are described elsewhere in the present teachings, particularly Section II.

The optical element can have any suitable physical property (or properties) related to any suitable optical characteristic of the element. The physical property can be a dimension-related property (such as volume, thickness, area, etc.), shape, swellability, etc. The optical characteristic can be any suitable aspect of interaction with electromagnetic radiation (light), including the spectrum, direction, intensity, and/or pattern of radiation that is refracted, reflected, absorbed, emitted, polarized, diffracted, and/or the like. In some embodiments, the optical characteristic is measured by illumination of the optical element with visible light.

Optical elements can be fabricated by any suitable methods. In an exemplary approach, the optical elements are fabricated with a sheet or plate of photographic emulsion or other photosensitive material. The sheet can be illuminated with radiation from a laser (or other suitable (e.g., coherent) light source) that is transmitted through the sheet from the laser and then reflected back through the sheet from a mirror, to create interference fringes corresponding to alternating layers of increased and decreased light exposure. The exposed sheet can be chemically processed, for example, developed, fixed, and/or bleached, so that the fringes are recorded in the sheet as layers of altered optical character.

Further aspects of holograms for detection of analytes, including fabrication and use of the holograms, are described in the following patent, which is incorporated herein by reference: U.S. Pat. No. 5,989,923, to Lowe et al., issued Nov. 23, 1999.

II. Nucleic Acids

The system of the present teachings detects the presence/absence and/or amount of a nucleic acid analyte, generally using a nucleic acid probe disposed in an optical element. A nucleic acid (or an oligonucleotide, an oligomer, or a polynucleotide), as used herein, is a polymer of at least two nucleotide subunits linked together. The nucleic acid can be single-stranded or at least partially double-stranded (duplex), among others. Double-stranded nucleic acids generally are formed by hydrogen-bonding (base-pairing) between aligned nucleotides of paired strands of nucleic acids, for example, adenosine (A) paired with thymidine (T) (or uridine (U) in RNA), and guanosine (G) paired with cytidine (C), among others. The nucleic acids also can be at least partially triple-stranded, such as formed by Hoogstein base pairing.

The nucleic acid analyte and/or probe can have any suitable natural and/or artificial structure. The nucleic acid can include a phosphodiester backbone such that the nucleic acid has a negative charge in aqueous solutions of neutral pH. A phosphodiester backbone generally includes a sugar-phosphate backbone of alternating sugar and phosphate moieties, with a nucleotide base (generally, a purine or a pyrimidine group) attached to each sugar moiety. Any sugar(s) can be included in the backbone including ribose (for RNA), deoxyribose (for DNA), arabinose, hexose, 2′-fluororibose, and/or a structural analog of a sugar, among others. The nucleotide base can include, for example, adenine, cytosine, guanine, thymine, uracil, inosine, 2-amino adenine, 2-thiothymine, 3-methyl adenine, C5-bromouracil, C5-fluorouracil, C5-iodouracil, C5-methyl cytosine, 7-deazaadeine, 7-deazaguanine, 8-oxoadenine, 8-oxoguanine, 2-thiocytosine, or the like. The nucleic acid analytes and/or probes of the present teachings can be analogs including any suitable alternative backbone. Exemplary alternative backbones can be less negatively charged than a phosphodiester backbone and can be substantially uncharged (neither positively nor negatively charged). Exemplary alternative backbones can include phosphoramides, phosphorothiozates, phosphorodithioates, O-methylphosphoroamidites, peptide nucleic acids, positively charged backbones, non-ribose backbones, etc. Nucleic acids with artificial backbones and/or moieties can be suitable, for example, to increase or reduce the total charge, increase or reduce base-pairing stability, increase or reduce chemical stability, to alter the ability to be acted on by a reagent, and/or the like. In exemplary embodiments, nucleic acid probes (such as peptide nucleic acids) with a reduced negative charge can be employed with phosphodiester-based analytes to increase the sensitivity of optical elements for detection of the analytes.

A. Analytes

An analyte, as used herein, is a nucleic acid that is the subject of an analysis performed with an optical element. The analyte can be from any suitable source, can have any suitable structure, and can be analyzed for any suitable feature. In some examples, the analyte can be a template, that is, a nucleic acid used as a model or guide for copying the analyte with amplification reagents, to amplify the analyte.

The analyte can be from any suitable source. Exemplary sources can include a human subject, a nonhuman animal, a plant, a microorganism, a research sample, a forensic sample, an environmental sample (such as soil, air, water, etc.), a food sample, and/or in vitro synthesis, among others.

The human subject can be a disease patient, a genetic screening subject, a person to be identified, a forensic subject, and/or the like. The analyte can be obtained from any suitable site in the human subject, including a sample from blood, plasma, serum, sperm, urine, fecal matter, vomit, sweat, tears, sputum, mucus, milk, a tissue sample, a tumor biopsy, cultured cells, and/or the like.

The analyte can be obtained in any suitable form by any suitable processing. The analyte can be disposed in fluid, such as an aqueous sample. Alternatively or in addition, the analyte can be disposed on a surface, for example, the sample can be collected by swiping or swabbing the surface with an optical element and/or a test device including the optical element. The analyte can be analyzed from a sample as collected, or the sample can be processed into a crude lysate or the analyte can be purified from the sample. Exemplary methods of purification can include ion exchange chromatography, selective precipitation, and/or centrifugation, among others. The analyte can be single- or double-stranded and can have any suitable size. In some examples, the analyte can have a single size or a set of sizes produced by shearing, restriction endonuclease digestion, in vitro synthesis, amplification (copying), limited chemical digestion, and/or the like. In some examples, the analyte can be a plurality of discrete strands of similar or identical length and sequence content, or of distinct lengths and/or sequence content. Strands of distinct length can be overlapping fragments, for example, including a common region of similar or identical sequence, such as for hybridization (base-pairing) with a probe. The analyte can be any suitable size relative to the probe. In some examples, the analyte is about the same size as the probe. In some examples, the analyte is longer than the probe and can be substantially longer than the probe, such as at least about two, ten, or one hundred times as long as the probe.

The analyte can include copies of a parent analyte in a sample produced by amplification of the parent analyte. The analyte copies can correspond to a selected region of the parent analyte defined by one or more amplification primers. Accordingly, the analyte copies can be of any suitable length relative to the parent analyte. The parent analyte and/or the analyte copies can be DNA and/or RNA. For example, the parent analyte can be DNA and the analyte copies RNA, the parent analyte RNA and the analyte copies DNA, or both the parent analyte and the analyte copies can be DNA (or both RNA). Amplification can be performed isothermally and/or with thermal cycling (e.g., with the Polymerase Chain Reaction (PCR)), among others, and can be exponential or linear. Isothermal amplification, as used herein can include or lack an initial thermal denaturation to denature nucleic acid duplexes in a sample. Exemplary methods of isothermal amplification thus can include Nucleic Acid Sequence-Based Amplification (NASBA), Loop-Mediated Isothermal Amplification (LAMP), Rolling Circle Amplification (RCA), Self Sustained Sequence Replication (S3R), Strand Displacement Amplification (SDA), and/or the like.

B. Probes

A probe, as used herein, is a selective binding partner for a nucleic acid analyte. The probe can be from any suitable source and can have any suitable structure.

The probe can be obtained from a natural and/or artificial source. Accordingly, the probe can be synthesized or formed by a cell(s), a cell lysate(s), a synthetic enzyme(s), chemical synthesis, enzymatic cleavage, chemical cleavage, and/or ligation, among others. The probe thus can be RNA, DNA, or any suitable analog thereof. Furthermore, the probe can belong to the same structural class of molecules as the analyte (e.g., each being DNA or each being RNA) or to a different class of molecules (e.g., the probe being a nucleic acid analog and the analyte being RNA or DNA, among others).

The probe can have any suitable backbone structure relative to the analyte. In some examples, the probe can have a different backbone than the analyte, such as a less charged backbone in the probe and a more charged backbone in the analyte (or vice versa). With this arrangement, the analyte can have a greater effect than the probe on the charge of the optical element, and thus, for example, a greater ability to bring ordered water molecules into the optical element (and a greater effect than the probe on the volume of the optical element). In exemplary embodiments, the probe can have an analog (non-phosphodiester) backbone, and the analyte can have a conventional (phosphodiester) backbone. The analog backbone of the probe can lack phosphate moieties, ribose moieties, or both. In some examples, the analog backbone of the probe can include a plurality of amide moieties. In some examples, the analog backbone can be a peptide backbone, such that the analog is a peptide nucleic acid. A peptide backbone, as used herein, is any backbone that can be hydrolyzed to release a plurality of amino carboxylic acids, particularly alpha-amino carboxylic acids. In exemplary embodiments, the peptide nucleic acid has a backbone formed of linked N-(2-aminoethyl)-glycine subunits, which position an array of nucleotide bases through methylene carbonyl moieties of the backbone.

The probe can be configured to form a duplex with the analyte through base-pair interactions, so that the probe and analyte together form an at least partially double-stranded nucleic acid. Accordingly, a section (or all) of the probe can be complementary to a section (or all) of the analyte. Alternatively, or in addition, the probe can include a double-stranded region, independent of the analyte, for example, to couple the probe to the matrix of the optical element. The probe can be configured to hybridize (base-pair) to any region of the analyte, for example, the probe can hybridize adjacent an end or spaced from the end of the analyte.

The probe can include a moiety that facilitates covalent linkage or noncovalent association with the matrix of the optical element. The moiety can include a member of a specific binding pair (such as biotin, avidin, an epitope, a ligand, etc.) and/or a chemically reactive moiety that can react covalently with the matrix and/or subunits thereof. The moiety can be incorporated into the probe during and/or after probe synthesis.

The system of the present teachings can provide an optical element containing only one probe or a plurality of probes. The plurality of probes can be configured to form duplexes with different analytes and/or different regions of the same analyte, among others. In some examples, distinct probes can be disposed in distinct regions of the optical element.

III. Contacting Optical Elements with Samples

Samples and optical elements can be contacted in any suitable compartment or vessel, under any suitable conditions, and with or without any suitable amplification of the analyte. Contacting an optical element with a sample being tested for a nucleic acid analyte (and/or contacting a sample with an optical element) can be produced by relative movement of the optical element and the sample. Accordingly, contacting can be produced by moving the optical element into engagement with a static sample, moving the sample into engagement with a static optical element, or moving both the sample and the optical element into contact with one another.

Analytes and optical elements can be contacted in a compartment or vessel of any suitable structure. The compartment and/or vessel can be defined by a bottom wall, side walls, a top wall or cover, and/or the sample fluid, among others. Exemplary compartments or vessels thus can include (or be disposed adjacent or within) platforms (with no side walls or cover), wells (no cover), or fully enclosed vessels (with side walls and a cover).

The compartment and/or vessel can have any suitable shape and size. Exemplary shapes that can be suitable include sheet-like, cylindrical, frustoconical, semi-spherical, and/or polyhedral (e.g., a parallelepiped), among others. The compartment and/or vessel can have a volume that is substantially larger than the volume of the optical element (e.g., at least about two or five times the volume of the optical element), so that the optical element and the sample can be received in the compartment. In exemplary embodiments, the volume of the compartment and/or vessel can be less than about 500, 100, 20, or 5 microliters. In some examples, a support or backing for the optical element defines a wall of the compartment and/or vessel. Accordingly, the compartment and/or vessel can have an area that corresponds to the area of the optical element. Alternatively, the compartment and/or vessel can be larger in area than the optical element. Furthermore, the compartment and/or vessel can have a height or thickness that is substantially greater than the thickness of the optical element, for example, at least about twice, three times, or five times greater than the optical element thickness.

The compartment and/or vessel can be formed of any suitable material(s). The material can substantially inert to reaction with nucleic acids. Furthermore, the material can be nonporous, to restrict flow of fluid, and/or porous to permit flow. In some examples, the compartment and/or vessel can be constructed of a combination of different materials to provide selective fluid retention or flow in different regions of the compartment/vessel. Exemplary materials that can be suitable for construction of the compartment/vessel can include plastic, glass, ceramic, cellulose (e.g., paper), and/or the like.

The step of contacting can be performed with any suitable sample volume relative to the volume of the optical element. In some embodiments, the sample has a volume of at least about two, three, five, or ten times the volume of the optical element, to reduce background resulting from volume-based partitioning of sample-based non-analyte nucleic acids (or other nonspecific sample components) into the optical element without specific binding. However, in some embodiments, this background can be reduced by washing the optical element with a fluid lacking the non-analyte nucleic acids and/or other nonspecific sample components after the step of contacting, permitting the use of a smaller volume of sample (e.g., optionally less than twice the volume of the optical element).

The step of contacting can be performed at any suitable temperature(s). The temperature can be substantially constant or can be varied, such as to denature double-stranded duplex and permit probe-analyte hybridization, to adjust the binding stringency, to promote amplification (isothermal or with thermal cycling), and/or the like. Amplification can be performed at any suitable time including before and/or during the step of contacting.

The step of contacting can be performed in the presence of any suitable reagents. Exemplary reagents include reagents for amplification (such as a polymerase, nucleotide substrates (e.g., dNTPs or NTPS), enzyme cofactors, salt, a reducing agent, and/or the like) and/or reagents to adjust binding stringency or rate (such as an organic solvent, salt, a chaotropic agent, an exclusion polymer (such as polyethyleneglycol), and/or the like).

The optical element and/or sample can be treated after contacting with the sample. Exemplary treatment can include removal of the sample from adjacent the optical element and/or one or more washing steps (placement of wash solutions in contact with the optical element). Washing steps can be suitable to, for example, adjust the ionic strength of the fluid within the optical element (such as to remove excess salt) and/or to remove amplification reagents and/or unbound nucleic acid, among others. Exemplary wash solutions can include water and/or aqueous salt solutions, with or without a pH buffer, among others.

IV. Detecting Optical Characteristics of Optical Elements

An optical characteristic of the optical element can be detected before, during, and/or after contact with a sample being tested for a nucleic acid analyte. Detection can be performed with any suitable light sources and any suitable light sensors.

Any suitable light source(s) can be used to generate and/or reveal optical characteristics of the optical element. Exemplary light sources for detection of optical characteristics can include ambient light sources and/or directed light sources that can direct light selectively toward the optical element. Exemplary light sources thus can include the sun, diffuse indoor lighting, incandescent light sources, fluorescent light sources, hybrid incandescent-fluorescent light sources, arc lamps, light-emitting diodes, high-intensity discharge light sources, and/or lasers, among others. The light source, alone or in combination with a suitable optic, such as a filter, can direct light of any suitable wavelength or wavelength spectrum toward the optical element, including broad spectrum light (e.g., white light), and/or narrow spectrum light (e.g. light of a blue, green, and/or red wavelength, among others). The light can be polarized or nonpolarized and can be coherent or noncoherent. The light can be directed toward the optical element in a direction orthogonal, oblique, or parallel to a face of the optical element.

Any suitable sensor(s) can be used to detect interaction of the optical element with the light from the light source. Exemplary sensors can include the eye (for an optical characteristic that changes visibly), an electronic detector (e.g., a charge-coupled device (CCD), a photodiode or photodiode array, a photomultiplier tube, and/or the like), a chemical detector (e.g., film), and/or the like. Exemplary detectors can include point detectors (e.g., a photodiode and/or photomultiplier tube) and/or image detectors (e.g., a CCD) among others. The sensor can be positioned for detection at a predefined angle(s) to the optical element, can scan through a range of angles, and/or the like.

A change in an optical characteristic of the optical element can be determined by any suitable criteria. If detected by eye, the change can be a perceived change, such as a perceived, generally qualitative, change in color, brightness, and/or pattern, among others. If detected with an instrument, the change can be quantified to determine the magnitude of the change. In some examples, the optical characteristic can be compared with an expected or calculated value, an initial (measured) value, and/or one or more values obtained using one or more other optical elements that serve as a control.

### V. EXAMPLES

The following examples describe selected aspects and embodiments of the present teachings, particularly apparatus and methods for detection of nucleic acids using holographic sensor elements. These examples are included for illustration and are not intended to limit or define the entire scope of the present teachings.

### Example 1

### Vessels for Contacting Samples and Optical Elements

This example describes exemplary vessels that can be suitable for contacting samples and optical elements to allow detection of nucleic acid analytes; see FIGS. 3 and 4.

FIG. 3 shows an exemplary vessel 80 for contacting an optical element 82 with a sample 84 being tested for a nucleic acid analyte 86. The vessel can configured as a well with a bottom wall 88, side walls 90, and a removable cover 92. The vessel can be at least partially transparent, to permit detection of the optical element through the bottom wall (and/or side walls and/or cover) of the vessel. Furthermore, the vessel can be connected to other vessels, as in a multi-well microplate, to a handle, and/or can be an isolated structure, among others.

The optical element can be disposed horizontally in the vessel. For example, the optical element can be formed in the vessel, in abutment with the bottom wall. Alternatively, the optical element can be introduced into the vessel after the optical element has been formed.

The cover can reduce evaporation of the sample (and/or loss of fluid from the optical element). Accordingly, the cover can permit heating the contents of the vessel, either once or repetitively (such as for amplification by the polymerase chain reaction). In some examples, the cover can be, for example, foil or a plastic sheet, among others.

FIG. 4 shows another exemplary vessel 100 for contacting an optical element 102 with a sample 104 being tested for a nucleic acid analyte 106. The vessel can configured generally as vessel 80 of FIG. 3. However, optical element 102 can be a removable structure placed into the vessel. Accordingly, the optical element can include a backing or support 108 that gives the optical element mechanical stability and/or permits its manipulation by a user. In some examples, the backing can be thicker than the optical element. The optical element thus can be placed into the vessel in any suitable orientation, such as a generally vertical orientation, as shown here, or a horizontal orientation as in FIG. 3, among others. Changes in the optical element can be detected with the optical element disposed in the vessel and/or after removal of the optical element from the vessel.

### Example 2

### Plates for Contacting Samples and Optical Elements

This example describes exemplary plates configured as test strips for contacting samples and optical elements to allow detection of nucleic acid analytes; see FIGS. 5 and 6.

FIG. 5 shows an exemplary test strip 120 for contacting an optical element 122 with a sample 124 being tested for a nucleic acid analyte. The strip can include a support member 126 in the form of a plate to which optical element 122 is connected. The support member can be configured as a handle and/or a backing for the optical element. A drop 128 of the sample can be placed in contact with the optical element such that the drop (via surface tension), the optical element, and flanking surfaces 130 of the support member define a compartment for contacting the analyte with the optical element. In some examples, the support member can provide side walls to restrict lateral flow of the sample and/or define a volume of sample to be contacted with the optical element. The support member can have any suitable thickness, including a thickness substantially greater than the optical element.

FIG. 6 shows another exemplary test strip 140 for contacting an optical element 142 with a sample being tested for a nucleic acid analyte. The strip can include a support member 144 and a porous member 146, such as a filter element, extending over the optical element. The porous member can include a plurality of pores 148 (represented schematically) that receive the sample from outside the strip and channel the sample to the optical element. In some embodiments, the support member can be transparent to facilitate detection of changes in the optical element from below the surface of the support member and opposite the optical element.

### Example 3

### Detection of Nucleic Acid Analytes with Increased Sensitivity

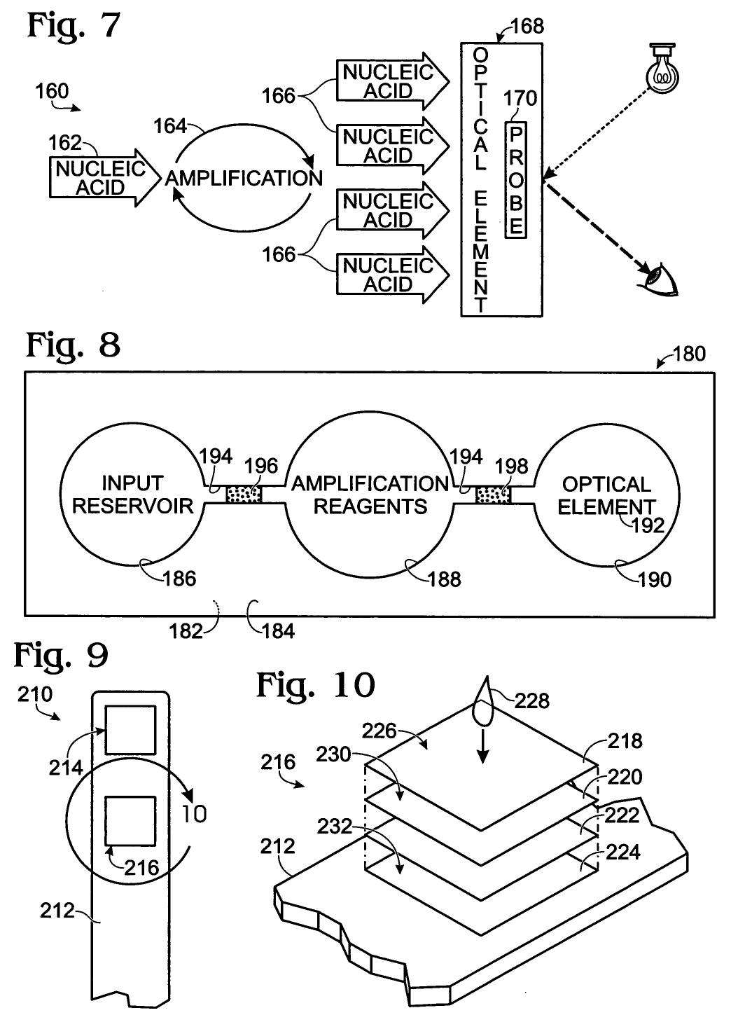

This example describes an exemplary method and exemplary assemblies for optical-element based detection of nucleic acid analytes with increased sensitivity using amplification reagents to copy the analytes; see FIGS. 7-10.

FIG. 7 shows an exemplary method 160 of detecting an amplified nucleic acid analyte. A parent analyte 162 can be amplified, shown at 164, by contact with amplification reagents, to produce analyte copies 166. The analyte copies can be contacted with an optical element 168 including a probe 170 that binds selectively to the analyte copies (and generally the parent analyte also), as described elsewhere in the present teachings. Amplification of the parent analyte (in a sample) to produce the analyte copies can be performed before and/or after the parent analyte (the sample) is contacted with the optical element.

FIG. 8 shows an exemplary apparatus 180 for performing method 160 of FIG. 7. Apparatus 180 can include a base 182 and a fluidic layer 184 disposed on the base to form a plurality of chambers. The chambers can include an input reservoir 186 to receive a sample, an amplification chamber 188 in amplification reagents for amplification of a parent analyte from the sample to create an amplified analyte, and a detection chamber 190 for contacting the amplified analyte with an optical element 192. The chambers can be connected by passages 194 containing valves 196, 198. The valves can be configured to provide a time-sensitive barrier to fluid flow between the chambers. In particular, the valves can be solid barriers that are configured to dissolve over time by contact with sample from an adjacent chamber. In some examples, the valves can be solid bridge valves formed, for example, by polyethylene glycol. In operation, a sample can be placed first in the input reservoir. Over time, the sample can dissolve first valve 196, permitting the sample to flow to the amplification chamber for contact with the amplification reagents. Parent analyte, if present in the sample, can be amplified in amplification chamber 188 to increase the copy number of the analyte. Over time, the sample can dissolve second valve 198, permitting the sample with its amplified analyte to contact the optical element and change an optical characteristic of the optical element. Accordingly, any change in the optical characteristic can be relatively rapid in relation to an alternative approach in which the analyte is amplified in contact with the optical element.

FIG. 9 shows another exemplary apparatus 210 for performing method 160 of FIG. 7. Apparatus 210 can be configured as a test strip having a support member 212 (which can also serve as a handle), and indicator regions 214, 216 connected to the support member. Apparatus 210 can be considered a layer-based implementation of apparatus 180 of FIG. 8.

FIG. 10 shows an exploded view of indicator region 216 of the test strip. Region 216 can include an upper valve layer 218, an amplification layer 220, a lower valve layer 222, and an optical element 224. The upper and lower valve layers can provide barriers between a sample input area 226 for receiving a sample 228, an amplification area 230 between the upper and lower valve layers, and an analyte detection area 232 disposed below the lower valve layer. Accordingly, the sample can contact the upper valve layer and dissolve the upper valve layer to gain access to the amplification reagents in the amplification area. Nucleic acid analyte in the sample can be amplified in the amplification area. The sample also can dissolve the lower valve layer so that the sample (and its amplified nucleic acid) can contact the optical element.

### Example 4

### Kits for Detection of Nucleic Acids

This example describes kits that can be provided by the present teachings.

Detection of nucleic acids can be conducted with kits. The kits can include an optical element(s) as described herein, a reagent(s) (e.g., an aqueous solution) for preparation of samples, a fluid transfer device(s) (such as a pipet), a light source, a detector, and/or instructions for use, among others.

The optical element(s) can be supplied in any suitable form. In some embodiments, the optical element can be provided as a gel or as a solid matrix that can be reconstituted into a gel by contact with fluid. The optical element can be supplied in a test device with any suitable support or frame connected to the optical element. In some examples, the optical element can be included in a test device formed as a plate (such as a strip). In some examples, the optical element can be connected to a frame that is substantially larger than the optical element, to provide a handle. The optical element can be supplied in an enclosure or package, to restrict loss of fluid from the optical element.

### Example 5

### Selected Embodiments

This example describes selected aspects and embodiments of the present teachings, presented as a series of numbered paragraphs.

1. A device to facilitate detection of a nucleic acid analyte in a sample, comprising an optical element having a patterned variation in local optical character and including an analog probe that selectively binds a nucleic acid analyte, the analog probe being an analog of a corresponding nucleic acid probe having a phosphodiester backbone, the analog probe having an analog backbone that is substantially less charged than the phosphodiester backbone of the corresponding nucleic acid probe, wherein the optical element has an optical characteristic created by the patterned variation in local optical character, and wherein the optical characteristic changes when the nucleic acid analyte binds the analog probe.

2. The device of paragraph 1, wherein the patterned variation includes periodic changes in the local optical character of the optical element along a line through the optical element.

3. The device of paragraph 2, wherein the local optical character is a refractive index of regions within the optical element.

4. The device of paragraph 1, wherein the optical element includes a photographic emulsion exposed to light disposed in an interference pattern and then chemically processed, and wherein the patterned variation corresponds to the interference pattern.

5. The device of paragraph 4, wherein the patterned variation is produced by local variations in size, number, and/or chemical structure of grains including silver in the photographic emulsion after chemical processing.

6. The device of paragraph 1, wherein the analog backbone is a peptide backbone.

7. A device to facilitate detection of a nucleic acid analyte in a sample, comprising an optical element structured as a hologram and including an analog probe that selectively binds a nucleic acid analyte, the analog probe being an analog of a corresponding nucleic acid probe having a phosphodiester backbone, the analog probe having an analog backbone that is substantially less charged than the phosphodiester backbone of the corresponding nucleic acid probe, wherein the hologram has an optical characteristic that changes when the nucleic acid analyte binds the analog probe.

8. The device of paragraph 7, wherein the hologram includes a record of an interference pattern, and wherein the record changes when the nucleic acid analyte binds the analog probe.

9. The device of paragraph 8, wherein the record is created by a spatial distribution of particles within the optical element.

10. The device of paragraph 9, wherein the spatial distribution includes patterned changes in particle size, number, and/or chemical structure within the optical element.

11. The device of paragraph 8, wherein the record is created by distinct layers within the optical element.

12. The device of paragraph 7, wherein the analog probe is a peptide nucleic acid.

13. A device to facilitate detection of a nucleic acid analyte in a sample, comprising an optical element including a matrix and an analog probe, the analog probe being configured to selectively bind a nucleic acid analyte and being an analog of a corresponding nucleic acid probe having a phosphodiester backbone, the analog probe having an analog backbone that is substantially less charged than the phosphodiester backbone of the corresponding nucleic acid probe, wherein the optical element has a physical property and an optical characteristic affected by the physical property, and wherein the physical property of the optical element changes when the nucleic acid analyte binds the analog probe, thereby changing the optical characteristic.

14. The device of paragraph 13, wherein the optical element is a gel.

15. The device of paragraph 13, wherein the matrix at least substantially includes gelatin.

16. The device of paragraph 13, wherein the optical element has an optical character that varies in a periodic fashion along a line through the optical element.

17. The device of paragraph 16, wherein the optical element includes particles having a property, and wherein the property of the particles varies in a cyclical fashion along the line.

18. The device of paragraph 13, wherein the optical property changes visibly when the nucleic acid analyte binds the analog probe.

19. The device of paragraph 18, wherein the optical element has a perceived color produced when the nucleic acid analyte binds the analog probe.

20. The device of paragraph 13, wherein the analog probe is a peptide nucleic acid.

21. The device of paragraph 13, further comprising a frame that holds the optical element and defines a compartment connected to or connectable with the optical element.

22. The device of paragraph 21, wherein the compartment includes reagents for amplification of the nucleic acid analyte.

23. The device of paragraph 21, wherein the frame defines an input site for receiving sample, and wherein the input site is separated from the optical element by a valve that can be dissolved by the sample.

24. The device of 21, wherein the frame is a strip configured to serve as a handle for manipulation by hand.

25. The device of paragraph 13, the physical property being a volume of the optical element, wherein the volume changes when the nucleic acid analyte binds the analog probe, thereby changing the optical characteristic.

26. A device to facilitate detection of a nucleic acid analyte in a sample, comprising an optical element including a matrix and an analog probe, the analog probe being a peptide nucleic acid disposed in the matrix and configured to selectively bind a nucleic acid analyte, wherein the optical element has a physical property and an optical characteristic affected by the physical property, and wherein the physical property of the optical element changes when the nucleic acid analyte binds the analog probe, thereby changing the optical characteristic.

27. The device of paragraph 26, the physical property of the optical element being volume, wherein the volume of the optical element changes when the nucleic acid analyte binds the analog probe, thereby changing the optical characteristic.

28. A method of detection of a nucleic acid analyte in a sample, comprising (A) contacting the device of any of claims 1-27 with a sample to be tested for the nucleic acid analyte such that the nucleic acid analyte, if present, alters the optical characteristic of the optical element of the device; and (B) detecting a change, if any, in the optical characteristic of the optical element produced by the step of contacting, thereby providing information about presence, absence, and/or amount of the nucleic acid analyte in the sample.

29. The method of paragraph 28, the optical element and the sample each having a volume, wherein the step of contacting is performed with the volume of the sample external to the optical element being substantially greater than the volume of the optical element.

30. The method of paragraph 28, further comprising a step of copying the nucleic acid analyte such that the nucleic acid analyte is amplified.

31. The method of paragraph 30, wherein the step of copying is performed after the step of contacting.

32. The method of paragraph 30, wherein the step of copying is performed before the step of contacting until a barrier separating the sample from the optical element in breached by the sample.

33. The method of paragraph 28, wherein a barrier separates the sample from the optical element before the step of contacting, and wherein the step of contacting is performed by the sample breaching the barrier.

34. The method of paragraph 28, wherein the step of detecting is performed by visual inspection of the optical element.

35. The method of paragraph 28, wherein the step of contacting includes a step of placing the optical element into a volume of fluid corresponding to the sample.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

We claim:

1. A device to facilitate detection of a nucleic acid analyte in a sample, comprising:
an optical element structured as a hologram and including a probe that is a peptide nucleic acid and that selectively binds a nucleic acid analyte, the hologram having an optical characteristic that changes visibly when the nucleic acid analyte binds the probe.

2. The device of claim 1, further comprising a frame that holds the optical element and that defines a compartment adjacent the optical element, wherein the compartment includes reagents for amplification of the nucleic acid analyte.

3. The device of claim 2, wherein the frame defines an input site for receiving a sample to test for the nucleic acid analyte, and wherein the input site is separated from the optical element by a valve that can be dissolved by the sample.

4. The device of 2, wherein the frame is a strip configured to serve as a handle for manipulation by hand.

5. A device to facilitate detection of a nucleic acid analyte in a sample, comprising:
an optical element having a patterned variation in local optical character and including an analog probe that selectively binds a nucleic acid analyte, the analog probe having an analog backbone that is substantially less charged than a corresponding phosphodiester backbone,
wherein the optical element has an optical characteristic created by the patterned variation in local optical character, and
wherein the optical characteristic changes when the nucleic acid analyte binds the analog probe.

6. The device of claim 5, wherein the patterned variation includes cyclical changes in the local optical character of the optical element along a line through the optical element.

7. The device of claim 5, wherein the local optical character is a refractive index of regions within the optical element.

8. The device of claim 5, wherein the optical element includes a photographic emulsion exposed to light disposed in an interference pattern and then chemically processed, and wherein the patterned variation corresponds to the interference pattern.

9. The device of claim 8, wherein the patterned variation is produced by local variations in size, number, and/or chemical structure of grains including silver in the photographic emulsion after chemical processing.

10. The device of claim 5, wherein the analog backbone corresponds to a peptide backbone.

11. The device of claim 5, wherein the optical element is a gel.

12. The device of claim 11, wherein the gel at least substantially includes gelatin.

13. The device of claim 5, wherein the optical characteristic changes visibly when the nucleic acid analyte binds the analog probe.

14. The device of claim 13, wherein the optical element changes color when the nucleic acid analyte binds the analog probe.

15. The device of claim 5, further comprising a frame that holds the optical element and that defines a compartment adjacent the optical element, wherein the compartment includes reagents for amplification of the nucleic acid analyte.

16. The device of claim 15, wherein the frame defines an input site for receiving a sample, and wherein the input site is separated from the optical element by a valve that can be dissolved by the sample.

17. The device of 15, wherein the frame is a strip configured to serve as a handle for manipulation by hand.

18. A method of detection of a nucleic acid analyte in a sample, comprising:
contacting the device of claim 5 with a nucleic acid analyte in a sample such that the nucleic acid analyte, if present, alters the optical characteristic; and
detecting a change, if any, in the optical characteristic produced by the step of contacting, thereby providing information about presence, absence, and/or amount of the nucleic acid analyte in the sample.

19. The method of claim 18, further comprising a step of copying the nucleic acid analyte such that the nucleic acid analyte is amplified.

20. The method of claim 18, wherein a barrier separates the sample from the optical element before the step of contacting, and wherein the step of contacting includes a step of breaching the barrier with the sample.
